(19) United States
(12) Patent Application Publication
(10) Pub. No.: US 2006/0122261 A1
(43) Pub. Date: Jun. 8, 2006

(54) [METHOD OF PREPARING LABDANE DITERPENE COMPOSITION, PREFERABLY ISOFORSKOLIN AND DEACETYLFORSKOLIN FROM FORSKOLIN EXTRACT AND THEIR USE FOR PROMOTING LEAN BODY MASS AND OTHER APPLICATIONS]

(76) Inventors: Muhammed Majeed, Piscataway, NJ (US); Rajinder Kumar Bammi, Bangalore (IN); Natarajan Sankaran, Bangalore (IN); Rajendran Ramanujam, Bangalore (IN); Satyan Kalkunte Seshadri, Bangalore (IN); Subbalakshmi Prakash, Piscataway, NJ (US)

Correspondence Address: SABINSA CORPORATION, 70 ETHEL ROAD WEST, UNIT 6, PISCATAWAY, NJ, 08854, US

(21) Appl. No.: 10/707,087

(22) Filed: Nov. 20, 2003

(51) Int. Cl. A61K 31/353

(52) U.S. Cl. 514/454

(57) ABSTRACT

The invention describes methods of preparation of isoforskolin extract, deacetylforskolin extract and related compounds from a natural source. Compositions and human and veterinary applications of such extracts in weight management through promoting lean body mass and in topical preparations for cellulite control, are also described. The compositions of the invention could be administered in a variety of dosage forms, including but not limited to ready-to-serve beverages, dry mixes for food products and beverages, oral preparations, injectables, emulsions, liniments or oils, to humans or animals.

### BACKGROUND OF INVENTION

1. Field of Invention

The invention describes methods of preparation of isoforskolin extract, deacetylforskolin extract and related compounds from a natural source. Compositions, and human and veterinary applications of such extracts in weight management through promoting lean body mass, and in topical preparations for cellulite control, are also described.

2. Description of Prior Art

Obesity has grown to epidemic proportions in many parts of the world. Obesity has been a major risk factor in number health disorders including cardiovascular diseases, diabetes, hypertension, cancer and others. Most weight loss pharmaceutical compositions and nutraceutical aids are designed to decrease the amount of body fat in an individual by decreasing the individual's appetite for food, decreasing the amount of food absorption in the individual, slowing down the rate of fatty acid synthesis within the body, or increasing the rate of catabolism of fatty acids.

The following are some examples of weight loss products and their mechanisms. Dexfenfluramine increases the brain levels of serotonin, a neurotransmitter and neurohormone that quells the appetite. Sibutramine also increases the levels of serotonin, as well as noradrenaline, and works to quell the appetite. Neuropeptide Y inhibitors curb the appetite, as well as stimulating the body to burn more sugars and less fat. Bromeriptine mimics the neurotransmitter dopamine, and may reduce blood sugar and fat production by the liver. Leptin, a hormone generated by adipocytes, affects the hypothalamus. Cholecystokinin, a hormone and neurotransmitter, acts to reduce appetite. Butabindide blocks an enzyme that inactivates cholecystokinin. Orlistat interferes with pancreatic lipase, which results in poor absorption of dietary fat. Insulinotropin is a glucagons-like hormone which prevents obesity by slowing down the emptying of the stomach. Bta-243 stimulates beta-adrenergic receptors on adipocytes, with a resulting increase in the burning of fatty acids. Troglitazone is a synthetic hormone which signals muscle cells to utilize fat for energy, rather than sugars. Cytokine regulators change the activity of hormone-like cytokines and alter the communication among cells, resulting in weight loss. Hydroxycitric acid acts as an inhibitor of enzyme citrate lyase, which subsequently slows down the synthesis of fatty acids and increases the rate at which fatty acids are burned.

The average amount of body fat in the American male is 22 to 25%, and in the American female, the average amount of fat is 33 to 35%. These values are far above optimal values, which are 15 to 19% for 20 to 29 year old males and 19 to 23% for 20 to 29 year old females. Corresponding values for 40 to 49 year olds are 17 to 21% and 21 to 25%, respectively; and for 60 year olds, the corresponding values are 19 to 23% and 23 to 27%, respectively. In highly overweight individuals, fat tissue can constitute up to 70% of body weight.

The remaining percentage of body composition corresponds to the lean body mass. Lean body mass is composed of muscle, vital organs, bone, connective and other non-fatty tissues in the body and most of the body water. The body's metabolic rate is in direct proportion to the amount of lean body mass. Therefore, considering the lean body mass is important for any weight loss strategy.

The aforementioned weight control means do not take into account the importance of maintaining or increasing the lean body mass in the process of weight loss. In fact, regimes to decrease body fat often contribute to the catabolic wasting of lean body mass. Increased lean body mass regulates body metabolism and helps in losing weight, as well as maintaining the accomplished weight reduction. On the other hand, diminished lean body mass slows down body metabolism and results in difficulties in maintaining an appropriate, healthy body weight. Thus, an ideal weight management approach should be to reduce body weight to acceptable levels by restoring the optimal proportions of fat to lean body mass. By maintaining or increasing the lean body mass while simultaneously reducing body fat, the weight loss regimen would serve the general purpose of improving the overall health of the individual.

Maintaining or increasing the lean body mass (for example, skeletal muscles) is one of the important considerations for any weight loss strategy because of lean body mass determines the rate of metabolism and the body's thermogenic response to food and food induced thermogenesis and the metabolic rate, in turn, control body weight by an increase in the catabolism of body fat. This is so because thermogenesis is preferentially fueled by fatty acids derived from stores of body fat and from food. In addition, a high rate of thermogenesis contributes to more food being absorbed and to the preferential build up of lean body mass rather than adipose tissue.

Forskolin (CAS no 66575-29-9) is a naturally occurring labdane diterpene from Coleus forskohlii (Bhat, S. V.; Ba-jwa, B. S.; Dornauer, H.; de Souza, N. J.; Fehlabar, H.-W.; Tetrahedron Lett., (1977), 18, 1669). It has several desirable pharmacological properties.

U.S. Pat. No. 5,804,596 revolutionized the concept of weight management by reporting the use of Forskolin composition from forskolin extract in promoting lean body mass and treating mood disorders.

Extracts from plants containing Forskolin is reported to contain related labdane diterpenes like Isofoskolin, deacetylforskolin, dideoxyforskolin, diacetylforskolins among others (de Souza N J J of Ethanopharmacology, 38, 1993).

Structure and activity studies have indicated the critical importance of concomitant nature of 1-α and 9-α hydroxy groups for activity. Studies have indicated that 7-deacetylforskolin and 6-acetyl-7-deacetylforskolin (isoforskolin) to be equally effective as Forskolin as hypotensive, anti-hypertensive, positive inotropic properties and as adenylate cyclase activator (R H Rupp, N J de Souza, A. N Dohadwalla in Forskolin: Its Chemical, Biological and Medicinal Potential, Hoechst India Limited , Bombay, 39-47, 1985)

Diverse biological activities are observed by raising the levels of cAMP and as a result activating protein kinase. Such properties have led to numerous uses of Forskolin. Due to such activities, more than 1500 citations dealing with the physiological properties of Forskolin and other synthetic derivatives appeared in Chemical Abstracts in 2001.

U.S. Pat. No. 4,476,140 describes a composition and method for treatment of Glaucoma by administration of a therapeutically effective amount of a material selected from the group consisting of forskolin, colforsin and polyoxygenated Labdane derivatives. The active agent concentration of 0.1% to 4% is reported herein to be physiologically effective when administered as a topical suspension to the eye.

U.S. Pat. Nos. 5,070,209, 4,978,678, 5,023,344, 4,871,764 describe novel 12-halogenated forskolin derivatives, intermediates and processes for the preparation thereof, and methods for reducing intraocular pressure utilizing compounds or compositions.

EP0268256 describes novel 12-halogenated forskolin derivatives, intermediates and processes for their preparation, and methods for reducing intraocular pressure utilizing the compounds or compositions.

Prior art does not teach or reveal any method of preparing of lsoforskolin or Deacetylforskolin composition from a extract of the Coleus forskohlli plant for nutraceutical or cosmeceutical applications, particularly in the area of weight management and cellulite. Such a method and compositions would be useful for providing a more pure form of isoforskolin and or Deacetylforskolin than that which is presently available on the market, and for providing a standardized amount of active labdane diterpene including isoforskolin and deacetylforskolin either alone or in combination with or without forskolin in ratios that occur naturally in extracts, which can thereafter be further processed by other manufacturers, or combined with nutritional supplements by end users.

### SUMMARY OF INVENTION

The invention describes the method of preparation and analysis of composition of labdane diterpenes, preferably, isoforskolin and deacetylforskolin either alone or in combinations with or without diterpenes such as Forskolin. The invention also describes the possible use of the aforesaid composition, orally in weight management preferably to promote lean body mass and reduce obesity. Another embodiment of the invention describes the use of such composition as anti-cellulite when applied topically in different dosage forms. The invention also describes the use of aforesaid composition with other known synthetic or natural phosphodiesterases, lipase inhibitors and other thermogenic extracts or molecules for weight management.

### DETAILED DESCRIPTION

Forskolin has the following structure

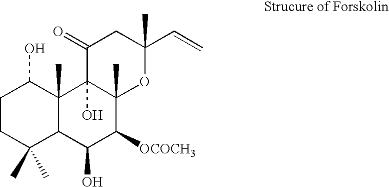

A closely related isomer is called lsoforskolin and it has the following structure

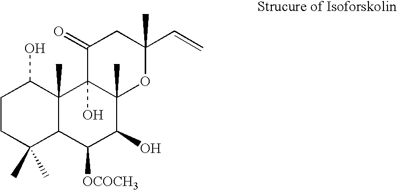

Another closely related structure of Forskolin is 7-deacetylforskolin whose structure is given below

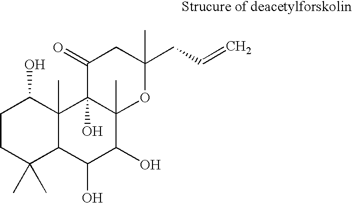

We believe that the invention compositions increases the lean body mass by stimulating the enzyme adenylate cyclase (AC), with a resulting increase in the levels of cAMP. The increase levels of cAMP in the tissues correspond well to enhancing the thermogenic response to food. An increase in the thermogenic response to food, in turn, improves absorption of nutrients and their preferential incorporation into lean body mass. Thus, the formation of lean body mass is promoted.

Isoforskolin and 7-deacetylforskolin have a mechanism of action similar to forskolin, in that, these molecules stimulate noradrenaline released from the sympathetic nerves to interact with beta-adrenergic receptors. This results in an increase in AC enzyme, with a subsequent rise in cAMP levels. cAMP stimulates the activity of protein kinase which phosphorylates a hormone-sensitive lipase to produce the active form of this enzyme. The lipase stimulates the release of fatty acids from body adipose depots. The released fatty acids stimulate the uncoupling process in the mitochondria, resulting in thermogenesis and provision of fuel to increase thermogenesis.

There is an increase in the beta-adrenergic dependent metabolic functions, which leads to an increase in the lean body mass, i.e., activation of phosphorylase in skeletal muscles, insulin secretion, and the synthesis and secretion of anabolic steroid hormones. lsoforskolin and Deacetylforskolin restores the level of monoamines for presynaptic availability, which has known anti-depressant action; There is an increase in cAMP in the postsynaptic effector cells in the brain, which is a “second messenger”, in comparison to the primary messenger” action of the monoamines.

In the present method of promoting lean body mass, the isoforskolin and/or deacetylforskolin should be administered in a daily dose of from about 10 to about 60 mg. It is preferred that the daily dose be divided into a plurality of individual doses. It is further preferred that three individual doses be used. In any case, the individual doses are preferably from about 10 to about 20 mg each.

In the present method of treating a mood disorder, the isoforskolin and deacetylforskolin should be administered in a daily dose of from about 10 to about 60 mg. It is preferred that the daily dose be divided into a plurality of individual doses. It is further preferred that three individual doses be used. In any case, the individual doses are preferable from about 10 to about 20 mg each.

In either method of the invention, the isoforskolin and deacetylforskolin can be administered in combination therapy with additional ingredients. Some examples of additional ingredients. Some examples of additional ingredients are extract from Garcinia cambogia in the form of natural (-) hydroxy citric acid or its salts (e.g., calcium or potassium salts); organic salts of vanadium (e.g., bis maltolato vanadium or bis glycinato vanadium); extract from Piper nigrum (black pepper) or Piper longum (long pepper) containing alkaloid piperine; or extract from Sida cordifolia containing alkaloid ephedrine or guarana extract or green tea extract.

The isoforskolin or deacetylyforskolin can be administered orally, topically or parenterally, although orally is preferred. Carriers, diluents or excipients are well known in the art.

The present invention includes isoforskolin and deacetylforskolin compositions. The composition can comprise about 1% to about 40% isoforskolin and/or 7-deacetylforskolin. It is more preferred to include about 5% to about 20% isoforskolin and/or 7-deacetylforskolin. It is even more preferred to include about 8% to about 15% isoforskolin and/or 7-deacetylforskolin. A composition containing about 10% isoforskolin and/or 7-deacetylforskolin is most preferred.

### EXAMPLE 1

### Commercial Process for Making Isoforskolin

The present invention also includes a method of preparing a isforskolin composition from a forskolin extract of Coleus forskohlii plant. The method involves extracting the pulverized roots of the plant with an solvent including water, C1-C4 alcohols, chlorinated solvents like MDC, toluene or hexane or solvent is a mixture of water and alcohol of which the preferred extracting medium is toluene. The concentrated toluene extract is precipitated with more non-polar solvents of the type heptane , pentane, hexane; filtered, and the filtrate is back extracted with mixtures of aqueous alcohols in ratios of 10:90 to 90:10 to obtain the desired molecule isoforskolin which is further crystallized with alcohols to obtain the desired purity.

### EXAMPLE 2

### Commercial Process for Making 7-deacetylforskolin

Total extract from the above example is dissolved in a solvent medium which includes but is not limited to alcohols, toluene or hexane and treated with immobilized enzyme lipase in concentrations of 0.1-10%, preferably 1-5%, at 37° C. under stirring for 12 hrs. Once the reaction is complete, the material is back extracted with mixtures of aqueous alcohols in ratios of 10:90 to 90:10 to obtain the desired molecule 7-deacetylforskolin which is further crystallized with alcohols to obtain the desired purity.

### EXAMPLE 3

### Commercial Process for Making Isofoskolin and 7-deacetylforskolin by Carbon Dioxide Extraction

Supercritical fluid extraction using carbon dioxide with and without entrainer such as ethanol, acetone or ethyl acetate extract Isofoskolin and deacetylforskolin from the roots of Coleus plant are described. The extracts were obtained at temperatures ranging from 25° to 120° C., preferably between 45°-55° C., the extraction fluid pressure was maintained between 100 to 300 bar preferably at 300 bars with or without co-solvents, preferably 5% to 80% ethanol, preferably 30-60% ethanol, for 1-5 hrs, preferably for 3 hrs and at carbon dioxide flow rate of 1-4 kg/h, preferably at 2 kg/hr. The extract obtained is hydrolyzed with lipase enzyme in a liquid media and crystallized out from ethanol to obtain deacetylforskolin of desired purity.

### EXAMPLE 4

### Comparative Effect of Isoforskolin, Deacetylforskolin and Forskolin Extract on Animal Model of Obesity

The study was done on Swiss Albino mice (Haffkine's Institute, Mumbai, India), aged between 25-30 weeks, on a total of 84 animals divided into 12 animals each.

Study Design: The mice under treatment were fed with diet rich in carbohydrates and fats. The diet produced reliable weight gain over controls. Drug treatment was started only when the difference between the body weights of control and the treated mice exceeded 10 g. The mice in respective groups, were given a daily fixed dose of Isofoskolin Extract, 10% D7-deacetylforskolinextract and Forskolin extract (1 mg/ml) for a period of six weeks, by means of gastric intubation, twice a day. At the end of sixth week, six mice per group were put to sleep after taking body weight and flab measurements. The blood was drawn by cardiac puncture and immediately centrifuged to separate the plasma and analyzed for Cholesterol, tryglycerides , glucose, Thyroxine T3, T4 and TSH levels.

Abdominal fat and thyroid were fixed for histopathology. A separate sample of adipose tissue were collected and analyzed for total lipid content. The remaining mice from each group were continued on the same diet as given during the first six weeks of treatment. This group was maintained in this manner for additional six weeks at the end of which non-invasive parameters such as body weight and flab were once again assessed.

Results: Both control and treatment mice tolerated the procedure of gastric intubation through out the six week treatment period. The extracts 10% Isoforskolin, 10% DAF and 10% Forskolin were well tolerated by the population of mice.

At the end of six weeks , six animals from each group, randomly selected, put to sleep and dissected to (a) excise adipose tissue and thyroid for histopathology (b) examine general anatomy and noting the changes, if any, in organs such as gut, heart, lungs, liver, pancreas, kidney, kidneys, reproductive organs etc. It was observed that none of the animals exhibited any abnormal anatomical features.

The abdominal fat shows interesting features in different groups: for example the control group mice under placebo or drug treatment showed good amount of abdominal fat distribution in bilateral lobes of adipose tissue. The animals from obese placebo group had significantly large quantity of abdominal fat with bilateral adipose tissue lobes extending into more than one third of the abdominal cavity. On the other hand, obese group animals treated with 10% Isofoskolin, 10% DAF and 10% Forskolin had practically exhausted the abdominal fat which was more pronounced in 10% Forskolin and 10% Isofoskolin group. In these groups of animals, there were significant loss of peritoneal fat as well as the fatty deposition in close association with uteri. Adipose tissue close to the kidney and ovary appeared to be adequate, though less in comparison with obese group under placebo treatment.

TABLE 1
Effect of treatment on the weekly record of body weights of experimental mice

| | Average body weight (n = 12) (in g) | | | | | | | |
|---|---|---|---|---|---|---|---|---|
| | | | | | | | | % Change between |
| Treatment | 0 Week | 1 Week | 2 Week | 3 Week | 4 Week | 5 Week | 6 Week | 0-6 weeks |
| Control- | 25.23 ± 2.80 | 26.26 ± 4.0 | 26.41 ± 3.28 | 26.18 ± 2.31 | 26.90 ± 3.08 | 27.4 ± 3.09 | 28.33 ± 2.64 | +12 |
| Placebo | | | | | | | | |
| Obese | 36.69 ± 2.71 | 37.72 ± 2.28 | 37.36 ± 2.29 | 37.18 ± 2.27 | 35.64 ± 2.69 | 35.5 ± 2.68 | 35.6 ± 3.10 | −0.003 |
| Placebo | | | | | | | | |
| Obese- | 37.18 + 3.48 | 36.81 + 3.25 | 34.93 + 3.80 | 33.00 + 4.54 | 31.86 + 4.09 | 31.69 + 4.66 | 29.61 + 5.77** | −20.37 |
| Extract 1 | | | | | | | | |
| Obese- | 36.11 + 3.21 | 36.32 + 3.55 | 35.08 + 3.88 | 34.36 + 4.01 | 32.15 + 3.91 | 31.87 + 4.81 | 30.78 + 5.96** | −14.76 |
| Extract 2 | | | | | | | | |
| Obese- | 37.33 ± 4.02 | 36.73 ± 3.67 | 34.88 ± 4.31 | 34.58 ± 3.76 | 32.67 ± 3.87 | 32.21 ± 3.34 | 31.54 ± 4.76** | −15.51 |
| Extract 3 | | | | | | | | |

**Significant reduction in weight by One Way Analysis of Variance (ANOVA) Extract 1: Contains 10% Forskolin, Extract 2: Contains 10% 7-deacetylforskolin, Extract 3: Contains 10% Isoforskolin. As seen in the above table, there is significant reduction in the total body weight in all the treatment group, the percentage change being maximum in Forskolin group.

TABLE 2
Effect of treatment on the weekly record of Abdominal flab of experimental mice

| | Average Abdominal Flab (n = 12) (in mm) | | | | | | | |
|---|---|---|---|---|---|---|---|---|
| | | | | | | | | % Change between |
| Treatment | 0 Week | 1 Week | 2 Week | 3 Week | 4 Week | 5 Week | 6 Week | 0-6 weeks |
| Control- | 0.81 + 0.12 | 0.91 + 0.09 | 0.92 + 0.10 | 0.93 + 0.09 | 0.94 + 0.11 | 0.97 + 0.12 | 0.99 + 0.11 | +22.22 |
| Placebo | | | | | | | | |
| Obese | 1.24 + 0.12 | 1.39 + 0.08 | 1.32 + 0.09 | 1.33 + 0.07 | 1.26 + 0.11 | 1.11 + 0.09 | 1.11 + 0.12 | −13.00 |
| Placebo | | | | | | | | |
| Obese- | 1.42 ± 0.15 | 1.27 ± 0.12 | 1.20 ± 0.14 | 1.11 ± 0.11 | 0.99 ± 0.11 | 0.91 ± 0.07 | 0.82 ± 0.11** | −42.22 |
| Extract 1 | | | | | | | | |
| Obese- | 1.38 ± 0.14 | 1.26 ± 0.17 | 1.22 ± 0.15 | 1.16 ± 0.12 | 1.08 ± 0.13 | 0.98 ± 0.11 | 0.88 ± 0.09** | −34.31 |
| Extract 2 | | | | | | | | |
| Obese- | 1.35 + 0.16 | 1.30 + 0.14 | 1.28 + 0.14 | 1.17 + 0.13 | 1.10 + 0.12 | 0.94 + 0.11 | 0.85 + 0.10** | −38.41 |
| Extract 3 | | | | | | | | |

**Indicates statistical significance in a One Way Analysis of Variance (AVOVA) Extract 1: Contains 10% Forskolin, Extract 2: Contains 10% 7-deacetylforskolin, Extract 3: Contains 10% Isoforskolin. As seen in the above table, there is significant reduction in the abdominal flab in all the treatment group, the percentage change being maximum in Forskolin group.

The extracts Isofoskolin 10%, 7-deacetylforskolin 10% and Forskolin 10%, when used at 2 mg/mice/day in divided dose significantly reduces the body weight and fat in mice.

All the treatment in obese animals show a significant reduction in the abdominal flab, in tandem with the weight loss. The flab dimension and weight gain or weight loss show a direct correlation.

The blood parameters such as glucose and cholesterol do not show statistically significant difference with the treatment at the end of six weeks treatment when compared with control or obese groups, suggesting that the treatments do not have any adverse influence on glucose or carbohydrate metabolism. The thyroid hormone T3 and TSH levels did not show a significant change in obese treated groups.

Histological characteristics of thyroid tissue appeared similar in control, obese and treatment groups.

The treatment does affect the quantity of fat stored in the adipose tissue. All the treatment groups have less density of adipose tissue and fat content when compared with controls and obese group. Apparently the treatment mobilizes fat stored in adipose tissue as a result the recipients lose obesity

1. A method of alleviating obesity and promoting lean body mass in a human individual in need thereof, comprising administering to the individual a lean body mass promoting composition containing effective amount of isoforskolin and or deacetylforskolin composition.

2. The method of claim 1. wherein the isoforskolin or deacetylforskolin is administered to a human or animal in need of such treatment in a dose of about 10 to about 100 mg in single or divided doses.

3. The method of claim 1. wherein the dosage could be oral including but not limited to tablets, capsules, powders, ready-to mix preparations, spansules, chewable, liquids, solutions, suspensions, beverage drinks, carbonated drinks, emulsions or topical including but not limited to o/w or w/o emulsions, lotions, oils, linament, aerosol sprays, implants among others.

4. The method in claim 1 can also be used in cosmeceutical preparations for controlling cellulite and for improving skin tone, texture and cellular rejuvenation.

5. The method in claim 1 also includes the commercial method of manufacture of isoforskolin and deacetylforskolin extract composition for nutraceutical and cosmeceutical applications.

6. The method in claim 5 sources the material from natural plant Coleus forskohlii and related species.

7. The method in claim 5 includes conventional solvent solvent extraction, and purification based on differential solubility in organic solvents.

8. The method in claim 5, particularly, the 7 deacetylforskolin is obtained by natural hydrolysis using lipase enzyme, or with use of any chemical methods for increasing the pH to 8 and above.

9. The method in claim 5 can also be obtained by liquid carbon dioxide extraction under supercritical temperature and pressure with the use of entrainer.
